(19) United States
(12) Patent Application Publication
(10) Pub. No.: US 2012/0172675 A1
(43) Pub. Date: Jul. 5, 2012

(54) INTEGRATED SYSTEM AND METHOD FOR SPECIAL NEEDS MANAGEMENT

(75) Inventors: PRADEESH THOMAS, SUNNYVALE, CA (US); PRIYA JAMES, MOUNTAIN VIEW, CA (US)

(73) Assignee: ALPHAVISTA SERVICES INC, Sunnyvale, CA (US)

(21) Appl. No.: 12/984,485

(22) Filed: Jan. 4, 2011

(51) Int. Cl. A61B 5/00; G06Q 50/00

(52) U.S. Cl. 600/300; 705/326

(57) ABSTRACT

The disclosure describes a method includes creating a special needs management tool on a computer based medium for a special needs assessment for a special needs individual using a special needs assessment format. In addition, the method includes recording the special needs assessment using the special needs management tool. The method also includes storing a special needs assessment report for a review by an authorized personal. The method further includes permitting the authorized person to access the assessment status.

### FIELD OF TECHNOLOGY

This disclosure relates generally to an integrated system and method for diagnosis, case management, treatment and analysis for special needs individual. The special needs management system is an integrated system. The system and method more specifically may be used by therapist, management authority, institutions, parents, special needs individual and stake holders for diagnosis, case management, treatment and analysis for special needs individual.

### BACKGROUND

“Special Needs” is an umbrella underneath which a staggering array of diagnosis can be envisioned. Individual with special needs may have mild learning disabilities or profound mental retardation; food allergies or terminal illness; developmental delays that catch up quickly or remain entrenched; occasional panic attacks or serious psychiatric problems. The designation is useful for getting needed services, setting appropriate goals, and gaining understanding for an individual and stressed family.

The special needs individual may have several issues such as medical issues, behavior issues, developmental issues, learning issues, and mental health issues, but may not be limited to these issues. Currently, each therapist have their own method to observe, evaluate, treat, document and produce a report. In organizations, the management and evaluators as officials have their own paper version of the file and allocate resources, schedule therapy, or assistance, validate the therapist in a disparate documentation method.

Currently, the progress report of the special needs individual is generated manually, there is no efficiency for tracking, and the reporting is subjective. There is no efficient process of managing the progress of the treatment. There are no integrated system currently to evaluate the performance of the therapist and the effectiveness of the used treatment method. There is a deficiency of an integrated tool for efficient management and overall report generation to provide effective therapy, time and budget management.

### SUMMARY

The disclosure describes a method, a system, a process and a server for assessing special needs individual requirements that may have medical issues, behavior issues, developmental issues, learning issues and mental health issues.

In one embodiment, a special needs management tool is used for performing a special needs assessment. In one embodiment, the special needs assessment is being recorded in real time by the therapist for evaluation and diagnosis.

In one aspect, a method includes generating a special needs management tool which includes an extensible computer process and providing an input guidance to available processors of an available computing environment. The method further includes automatically recording the special needs assessment and storing a special needs assessment report in real time. The method also includes automatically assembling a response from the stake holders, therapist, a government authority, a parent, a school administrator and a managed care personal using a distributed shared output.

The input guidance may be provided to each of the available access page for each involved individual and may be comprised of certain portions of the input data. The method may further include providing certain portions of the access to certain individuals. All the information may be stored in a database 150 for future reference.

In another embodiment, the therapist may access the special needs management system with access permission. In another embodiment, the therapist may use a method to create a special needs assessment test as a personalized test for each special needs individual.

The therapist, management, physician, parent, special needs individual and an administrator may create a special needs assessment report for review by each member of the care group. The report may be created, stored and viewed in real time in another embodiment.

In another embodiment, to comply with security rules, medical ethics rules and HIPPA rules access privilege may be enabled for each personal.

In another embodiment, a therapy library may be compiled by creating, populating from government, in-house and external sites. The therapy library may contain mandated, approved, suggested and validated test procedures, special needs assessment tests and other therapy related protocols, in one embodiment.

The methods, systems, and processes disclosed herein may be implemented in any means for achieving various aspects, and may be executed in a form of a machine-readable medium embodying a set of instructions. Other features and embodiments will be apparent from the accompanying drawings and from the detailed description that follows.

### BRIEF DESCRIPTION OF THE DRAWINGS

Example embodiments are illustrated by way of example and not limitation in the figures of the accompanying drawings, in which like references indicate similar elements and in which:

FIG. 1 is a system view illustrating a special needs management system, according to one embodiment.

FIG. 2 is an exploded view of the special needs module, according to one embodiment.

FIG. 3 is an exploded view of the therapist module, according to one embodiment.

FIG. 4 is an exploded view of the evaluator module, according one embodiment.

FIG. 5 is an exploded view of the management module, according to one embodiment.

FIG. 6 is an exploded view of parent module, according to one embodiment.

FIG. 7 is a diagrammatic system view of a computer device view in which any of the embodiments disclosed herein may be performed, according to one embodiment.

FIG. 8 illustrates a user interface view illustrating a log-in page of special education portal, according to one embodiment.

FIG. 9 is a user interface view illustrating a therapist dashboard, according to one embodiment.

FIG. 10 is a user interface view of a parent dashboard, according to one embodiment.

FIG. 11 is a user interface view illustrating a web page for the therapist to input clinical observation and behavior of the individual [patient] in a file associated with individual [patient], according to one example embodiment.

FIG. 12 is a user interface view illustrating a web page for the therapist to input object evaluation 1200 data associated with the special need individual, according to one example embodiment.

FIG. 13 is a continuation of FIG. 12 illustrating the web page to enable the therapist to input more objective evaluation data, according to one example embodiment.

FIG. 14 is a GUI view illustrating the brief summary of special need individual, according to one example embodiment.

FIG. 15 illustrates a method flow chart for providing a milestone development test for a special need individual.

FIG. 16 is a system view illustrating a special educating management system, according to one embodiment.

FIG. 17 is an exploded view of the special needs management server of FIG. 16, according to one or more embodiments.

Other features of the present embodiments will be apparent from the accompanying drawings and from the detailed description that follows.

### DETAILED DESCRIPTION

Several apparatus, methods and processes for an integrated system and method for special needs management are disclosed. Although the present embodiments have been described with reference to specific example embodiments, it will be evident that various modifications and changes may be made to these embodiments without departing from the broader spirit and scope of the various embodiments.

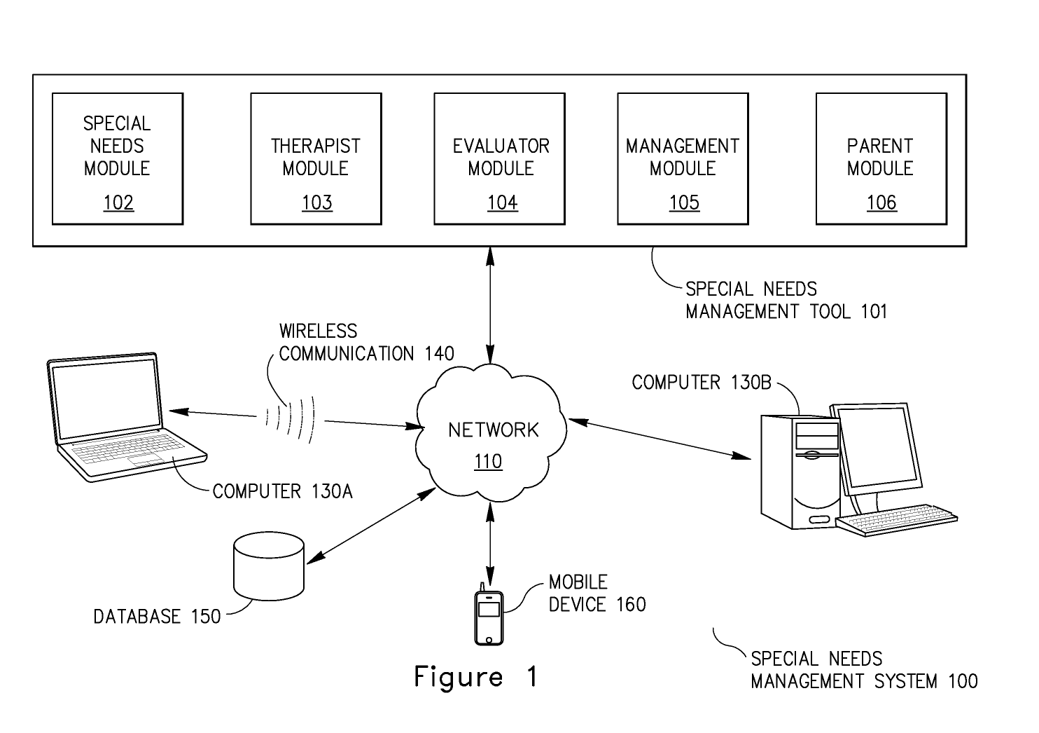

FIG. 1 is a system view illustrating a special needs management system 100, according to one embodiment. Particularly, FIG. 1 illustrates special needs management tool 101 supported by server, computers 130A-B, a network 110, a database 150, and a mobile device 160, according to one embodiment.

The special needs management system 100 may be designed to increase productivity, efficiency and accountability of various stake holders such as but not limited to therapist, management, evaluators, students and parents. The stake holders as described herein of the school education system may include at least one of therapist, parent and school administrator. The therapist 1602 may be teachers who are qualified and trained for training individual (e.g., children) requiring special training The special needs individual are the ones who may have one or more problems such as mental retardation, learning disability, speech language impairment, orthopedic impairment, traumatic brain injury, visual impairment, autism, dementia, Alzheimer's, deafness but not limited to the above problems.

In one or more embodiment, the special needs management tool 101 may be an integrated tool for the therapist, the physician, the management and the parent to create, view, annotate and store the information for the special needs individual. In one embodiment, the special needs management system 100 may be designed to reduce work load for the therapist 1602. In one embodiment, the special needs management system 100 may include the special needs management server (not shown) configured to track progress of the special need individual. The special needs management tool 101 may include a special needs module 102, a therapist module 103, an evaluator module 104, a management module 105, and a parent module 106. The special needs management system 150 as described herein may be designed to enable communication between the parents 1606, the therapist 1602 and the administrator 1604. The administrator may be a management personal or evaluator, but not limited to these personal.

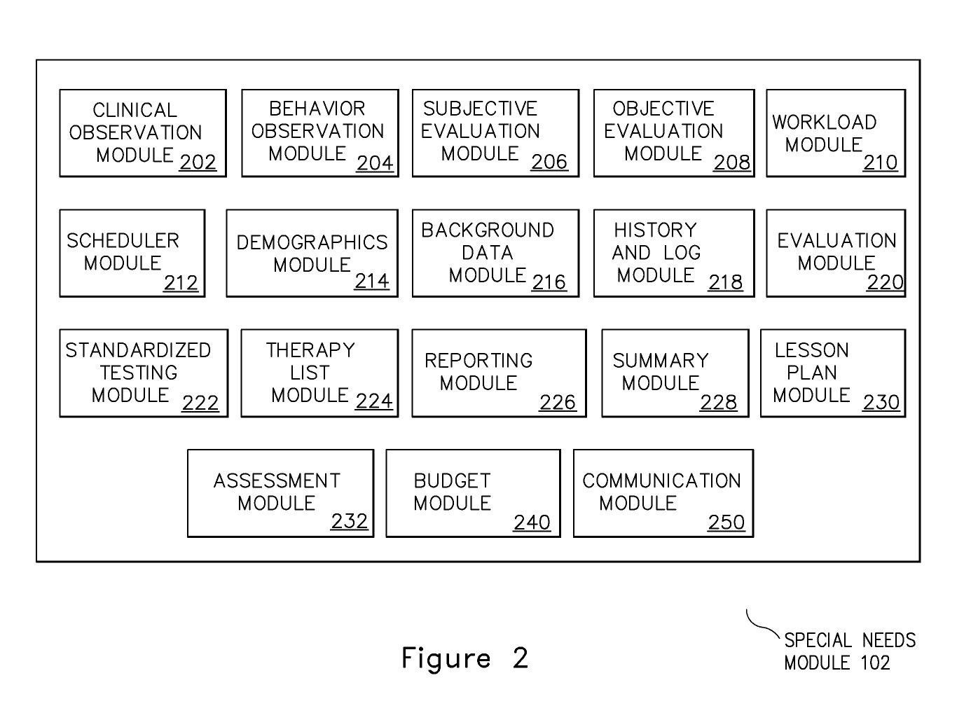

The special needs module 102 may be configured to manage all activities of the special needs management server. The special needs module 102 may be configured to provide a platform for the parents, administrator, and the therapist to interact amongst each other and to enable communication. The special needs module 102 is described in detail in FIG. 2.

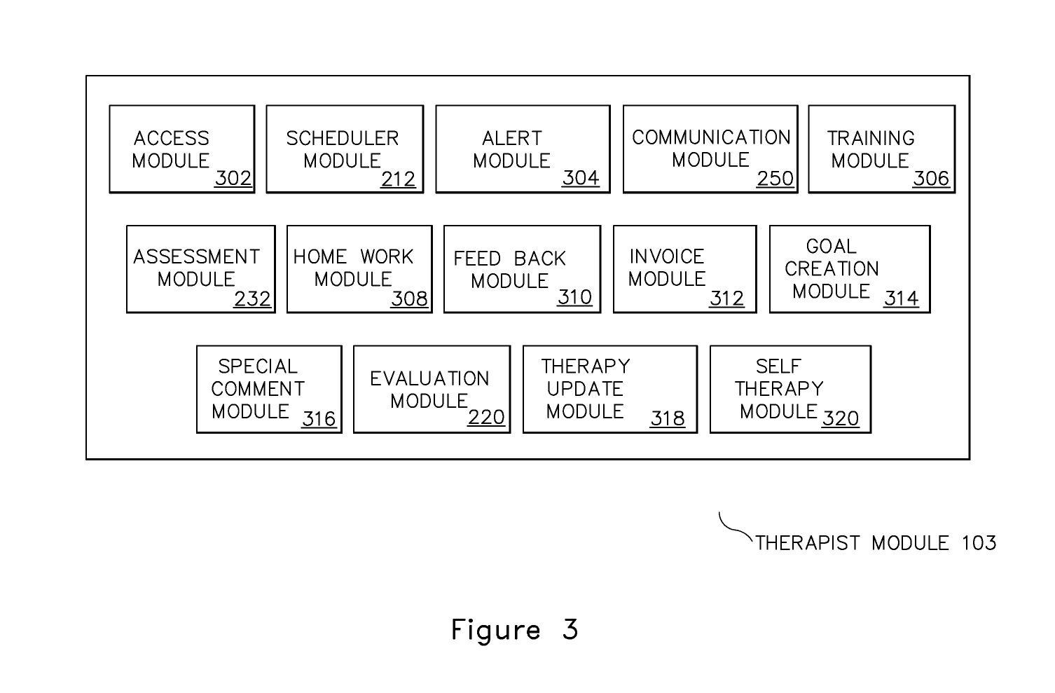

The therapist module 103 may be configured to assist a therapist to manage activities in organizations. In one embodiment, the activities of the therapist may include maintaining and managing schedules for a day, input information of therapies to be administered to the individual of special needs, providing homework to parents, input progress of special need individual, performing assessments, create lessons, provide online therapy, evaluating information obtained from the parents and other personal and generate reports. The therapist module 103 is described in detail in FIG. 3.

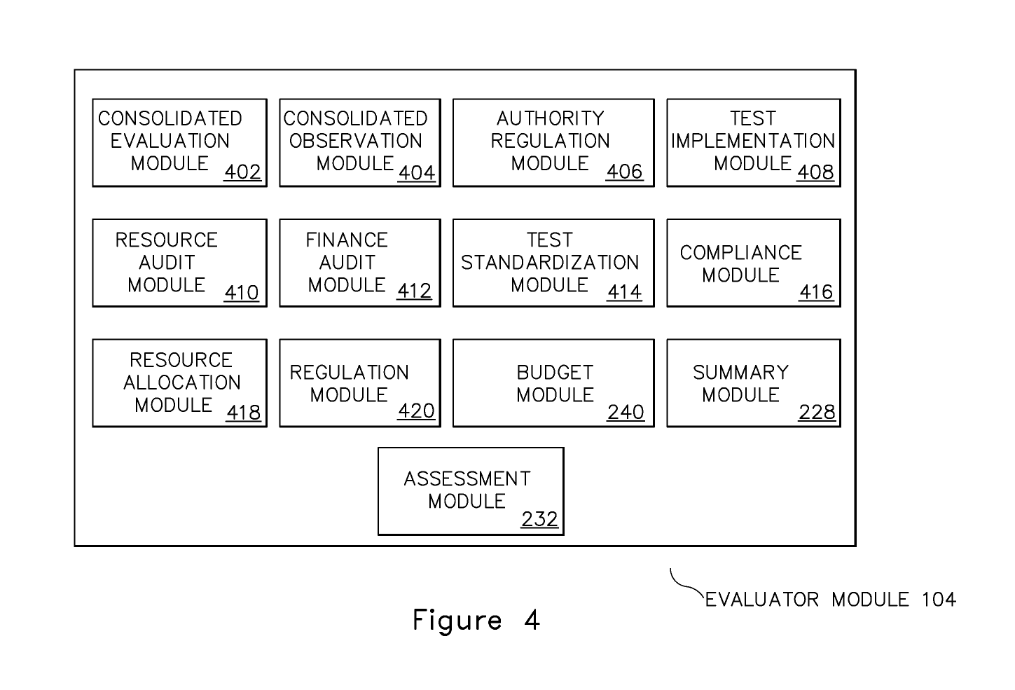

The evaluator module 104 may be configured to assist an evaluator or an administrator to manage the special needs management system 100, according to one embodiment. In one embodiment, the evaluator (hereinafter used as “administrator”) may be a government official, for example, district, county, state, federal and central administrator. The evaluator module 104 is described in detail in FIG. 4. In one of the embodiments, the administrator interacts with psychologist, physical therapist, speech language pathologist, occupational therapist, special educator, applied behavior therapist, parents, research specialist and teacher.

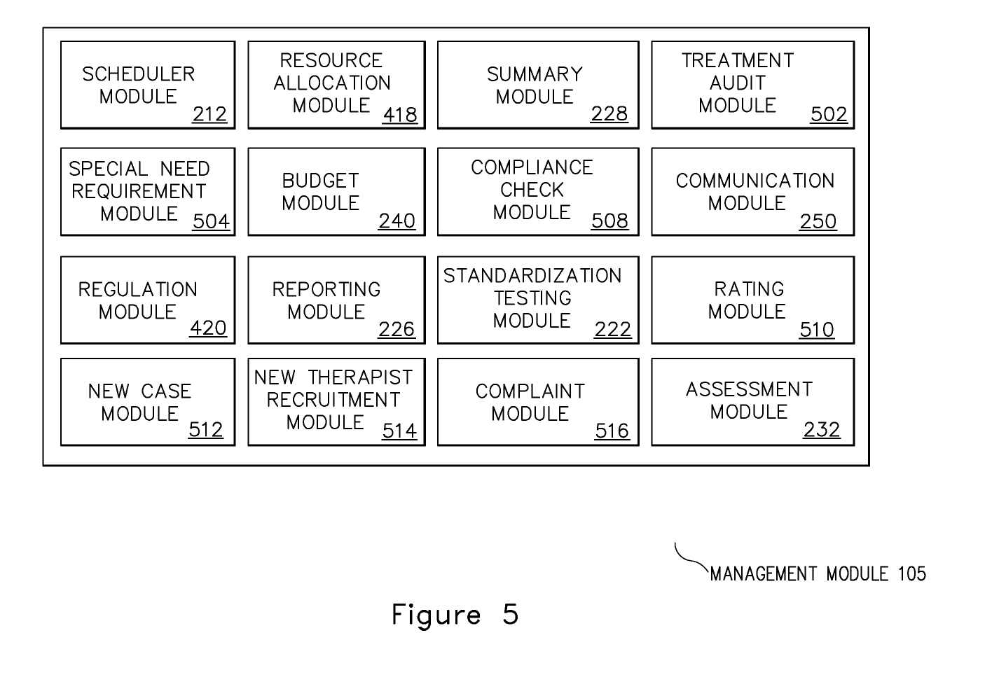

The management module 105 may be configured to manage the activities of the special needs management system 100. The activities may include, but is not limited to, tracking attendance of the therapist, performance of the therapist, tracking individual's progress, managing resources in special schools, providing alerts about lack of resources, and reporting absence of the therapist periodically. The management module 105 is described in detail in FIG. 5.

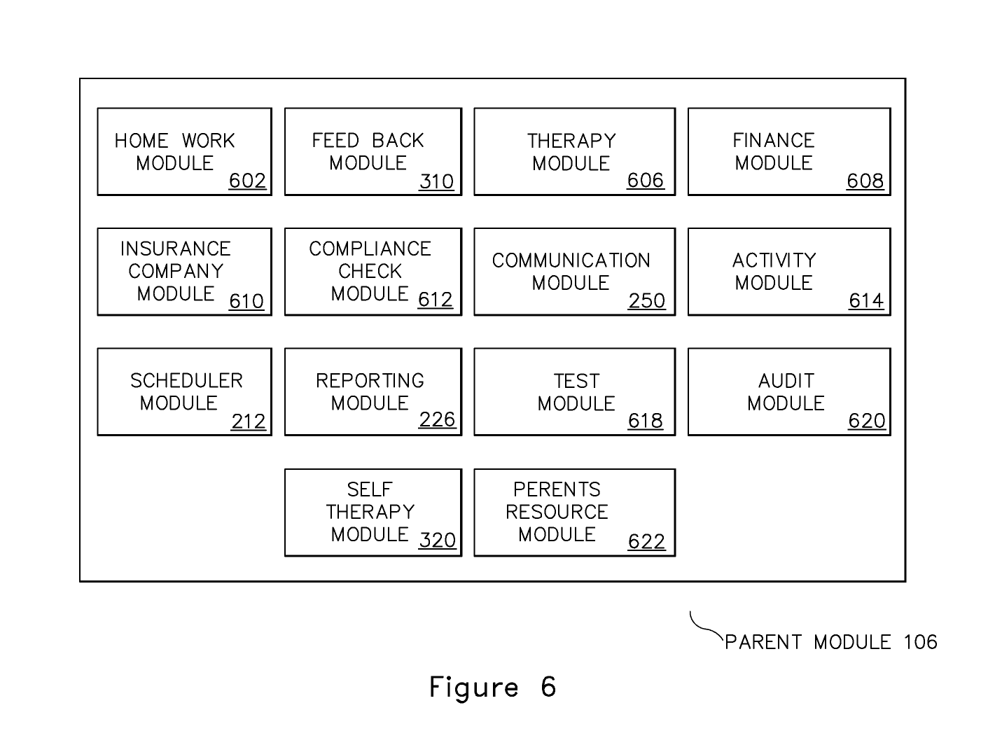

The parent module 208 may be configured to enable the parent o communicate with the therapist and the administrator. The parent module 208 may be configured to provide a platform to enable the parent to provide inputs and observations about the special needs individual to the therapist. The parent module 106 is described in detail in FIG. 6. Each of the modules as described above may be architecturally coupled to each other.

In one embodiment, the special needs management tool 101 may be accessed through any of the computer device 130_{A-B}. The special needs management tool 101 may be accessed through wired communication means or through wireless means. As illustrated, the special needs management tool 101 is accessed through the network 110, according to one embodiment. Each of the client device as described herein such as the computers 130_{A-B}, the mobile device 160 may include processors, operating system and a browser to enable viewing and operating interface views provided by the special needs management tool 101.

FIG. 2 is an exploded view of the special needs module 102, according to one embodiment. In particular, the special needs module 102 may include a clinical observation module 202, a behavior observation module 204, a subjective evaluation module 206, an objective evaluation module 208, a workload module 210, a scheduler module 212, a demographics module 214, a background data module 216, a history and log module 218, an evaluation module 220, a standardized testing module 222, a therapy list module 224, a reporting module 226, a summary module 228, a lesson plan module 230, an assessment module 232, a budget module 240, a communication module 250, according to one embodiment. In one embodiment, the special needs module 102 may be configured to integrate the other modules to enable proper functioning of the special needs management server.

In one embodiment, the special needs module 102 may be configured to support the special needs management system. In one or more embodiments, the special needs module 102 may be configured to provide an integrated platform for a therapist, a guardian and an administrator, users and stakeholders to provide input and to ensure progress of a special needs individual. In one embodiment, a special needs assessment test may be created that is personalized for testing each of the special needs individual by retrieving the special needs test from a therapy library. The therapy library may comprise of at least one of formal, informal assessment materials, therapy materials such as games and activities. The therapy library may be located and made accessible to the therapist from the database 150. In one embodiment, the special needs assessment test may be generated by an authority and downloaded to the therapy library. In some embodiments, the authority as described herein may be a government recognized medical doctor, a therapist, a government entity, a management personal, a parent, a managed care personal or a therapist assigned for treating the special need individual. In one embodiment, a treatment may be selected from the therapy library for the special needs individual. The treatment obtained from the library may be provided to the special needs individual. Once the treatment cycle is completed, the special needs individual may be evaluated after the treatment and a treatment result is generated which may be recorded. Furthermore, the condition, the report, and the treatment history may be stored in a file associated with the special needs individual and stored in the database 150. In all cases, the treatment method, and procedures are complied with a regulation rule. Also, the regulation rule may be a medical privacy rules, security privacy rules and special needs individual preference setting rule.

In one or more embodiment, the therapy library as described herein may be a compilation of several treatment method and the special needs assessment test suggested by the authority and approved by at least one of a physician, a psychologist, a physiotherapist, an audiologist, speech language pathologist and a parent. Also, the therapy library may be updated and upon improvement to the existing library on a periodical basis.

In one embodiment, a special needs assessment test may be generated using observing the special needs individual. In the current embodiment, the clinical observation module 202 and the behavioral observation modules 204 may be configured to generate templates to enable the therapist to record observations to generate special needs assessment test. The clinical observation module 202 may be an interface that is configured to be coupled to the therapist module 103 to enable the therapist to input clinical observation of the special needs individual for the physician, another therapist or a parent to review. The therapist may also be able to gather information about the special needs individual's clinical status provided by the physician to plan the therapy session. The clinical observation may be stored in the file associated with the special need individual. The behavior observation module 204 may be an interface that is configured to be coupled to the therapist module 103 to enable the therapist to input behavior observation of the special needs individual in a file associated with the special need individual. The subjective evaluation module 206 of the special needs module 102 may be an interface that is configured to be coupled to the therapist module 103 to enable the therapist to input subjective evaluation information of the special need individual. The subjective evaluation provided could be therapist own opinion, observation or perception on the progress of the special needs individual during treatment term. The therapist may provide this in a format to generate a report. The objective evaluation module 208 may be an interface that is configured to be coupled to the therapist module 103 to enable the therapist to input objective evaluation information of the special needs individual in the file associated with the special need individual. In one embodiment, the aforementioned module may be designed with standard options to enable the therapist to input text for convenience. The objective evaluation may be performed by using any formal tests specifically measuring academic and other skills. For example, the clinical observation module 202 may be configured to provide a GUI webpage to the therapist with standard options that is designed to capture the clinical observation information. In some embodiments, the therapist may be enabled with an option to input specific observations by allowing the therapist to create additional set of input forms.

The workload module 210 may be configured to manage the workload of the therapist. In some embodiments, the workload module 210 may be configured to report to the administrator regarding overload of work and a request for an additional therapist. The scheduler module 212 may be configured to enable the therapist to input schedules. For example, the scheduler module 212 may provide a GUI that includes calendar broken day wise and further broken by hour wise to enable the therapist to input the schedule, information for which the therapy is to be provided, type of therapy and the like. The demographics module 214, may include the ethinicity, race, geographical affiliation, dialect and language proficiency.

The background data module 216 may be configured to provide a platform for the therapist and the parent or caretaker to input background information associated with the special needs individual. The background data module 216 may include age, name, record number, personal history, educational history, family history, medical history and developmental history. The history and log module 218 may be configured to maintain the history and log information of each of the therapist, parents and the administrator, users and the stakeholders. For example, the history and log module 218 may store all the changes performed by the therapist or the users or the stakeholders on a special needs individual file.

The evaluation module 220 may be configured to assist the therapist to evaluate the observations to arrive at a conclusion of the therapy session of the individual with special needs. In some embodiments, the evaluation module 220 may be configured to track a milestone development of the special needs individual. The standardized testing module 222 may be configured to provide guidelines to the therapist to follow specific procedures to perform standard assessments on special need individual. The standardized testing module 222 may include, but not limited to, formal standardized assessment method, evaluation of the testing method and statistical scoring of the assessment method. The therapy list module 224 may be configured to provide a list of prescribed and federally approved therapies for a specific special needs individual. The reporting module 226 may be configured to assist the therapist, the parent and the administrator, the users, the stakeholders to generate reports at various stages of treatment. In some embodiments, special needs assessment report may be generated for a special needs individual using the special needs management tool 101. Further, the special needs assessment report may be generated using the special needs management tool 101 in a real time environment. Further, the special needs assessment report may be stored in the file of the special needs individual in the database 150 for a review by an authorized personal such as for example, a senior therapist, a doctor and the like. The stored special needs assessment report may be secured based on an access privileged rule.

The summary module 228 may be configured to enable a user of the system to generate reports associated with the special need individual. The lesson plan module 230 may be configured to assist the therapist to plan for the therapy at various stages. The budget module 240 may be configured to monitor the expenditure of the fund provided by the federal agencies to maintain the special needs management system. In some embodiments, the budget module 240 may generate and communicate reports to the administrator periodically. The communication module 250 in the special needs module 102 may be configured to enable communication between the therapist, the administrator and the parent. In one embodiment, the communication may be enabled by the communication module 250 through use of a standard protocol. In one example embodiment, the communication may be enabled through an email program, a chat program, or any other interactive enabling program.

FIG. 3 is an exploded view of the therapist module 103, according to one embodiment. In particular, the therapist module 103 of the special needs module may include an access module 302, a scheduler module 212, a therapy view module 304, a communication module 250, a training module 306, a homework module 308, a feedback module 310, an invoice module 312, a goal creation module 314, a special comment module 316, an evaluation module 220, a therapy update module 318, a self training module 320 and the assessment module 232, according to one embodiment. The therapist module 103 may be configured to assist the therapist in maintaining schedules and updating the special needs individual information. The therapist module 103 is described in detail below.

The access module 302 may provide and enable the therapist to access the therapist dash board. In one embodiment, the access module 302 may provide a graphical user interface to enable the therapist to access the therapist dash board. In one embodiment, each therapist may be provided with an account and a set of tools to enable the therapist to plan for a day. The planning process of the therapist may be supported in the therapist account through the scheduler module 212. The scheduler module 212 as described may be configured to enable the therapist to schedule for events in a day, a month and a year. In one example embodiment, the scheduler module 212 may provide a GUI that includes calendar, plan assistant and the like to support the therapist to plan without spending much time. In one embodiment, the scheduler module 212 may enable the scheduling a time slot for a care provider to meet the special needs individual on a needs basis

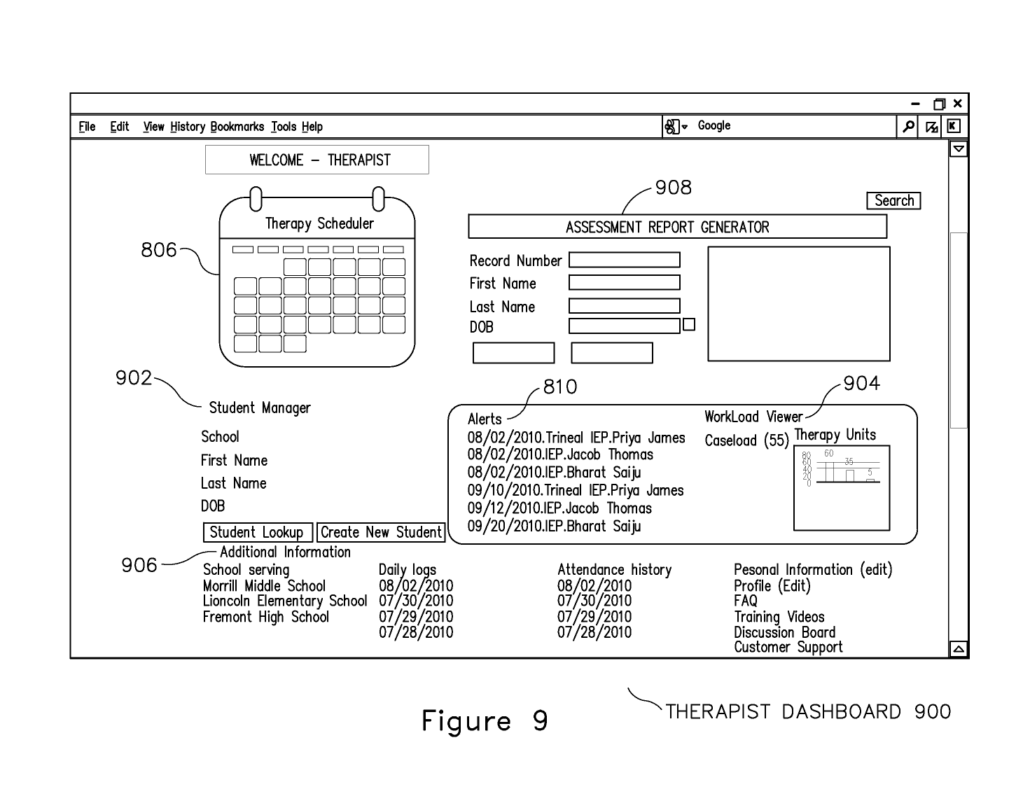

The therapist dash board and therapy scheduler is illustrated in FIG. 9. In one embodiment, the scheduler module 212 may provide a break up of time in a day. The therapist can input special needs individual information to link the file associated with the special needs individual in the database 150 in the scheduler to input training methodology for the special needs individual for a specific duration of time in a day. For example, morning 8 AM to 10 AM may be scheduled for training a child named William James, 10.30 AM to 1 PM may be scheduled for training a child named Christina and so on. In one embodiment, the schedule allotted may be further visually divided into an observation time, a training time, a test time, a report creation time and the like. The scheduler module 212 also prompts the therapist to provide a type of therapy that would be administered to the special needs individual in the specific schedule. For example, the therapy provided individually to the child William James may be a speech therapy to enable the child to rectify production of a /s/ sound as the goal.

In one embodiment, the therapy that would be administered to the special needs individual on a particular day being planned in advance may be viewed by the therapist and/or the parent through the therapy view module 304. In addition, the therapy view module 304 may also provide periodic alerts about the therapy that may have to be administered to a particular special needs individual on a particular day on a prescheduled time. The communication module 250 may enable the therapist to communicate the progress report of the special needs individual to the parent, administrator. In one embodiment, the communication module 250 may generate a readymade template based on the progress report which is graphically represented with charts and graphs associated with the special needs individual to be communicated to the parent/administrator. In one example embodiment, the communication template may be an email template.

The training module 306 may be configured to enable the parents to get trained on the therapy sessions and topics to be administered to the special needs individual. In addition, the training module 306 may also be configured to recommend training that may be suitable to the special needs individual based on the condition of the individual. The homework module 308 may be configured to generate a template to enable the therapist to input homework for the special needs individual to be communicated to the parent. In one embodiment, the homework maybe communicated to the parent of the child to instruct/guide the special needs individual to perform certain exercises, for example, spelling exercise for the /s/ sound. For example, in a case of assignment provided as a speech therapy for the /s/ sound, the parents may be given instructions and a set of words such as “Sat, Sir, Silly, beside, aside, Boston, mass, toes, and boss. The set of words may be provided as assignment for exercise purpose and for various other articulation reasons. The feedback module 310 may enable the therapist to provide feedback to the parents regarding the progress of the special need individual. The invoice module 312 may enable the therapist to bill for any materials or aid obtained externally to assist in improving the condition of the special need individual.

The goal creation module 314 may be configured to create goals for the special need individual. For example, a measurable goal for the correct production of the /s/ sound may be created saying “Child will produce /s/ sound in isolation with 80% accuracy in 10 consecutive trials”. The special comment module 316 may be configured to enable therapist to provide special comments such as suggestions, improvements etc. The evaluation module 220 may enable the therapist to input the results of the test, progress of the special needs individual etc. The therapy update module 318 may be configured to enable the therapist to update the therapy being administered or planned in advance based on the progress of the special need individual. The self-training module 320 may be configured to provide training to the special needs individual through parents. The assessment module 232 of the therapist module 103 may be configured to enable the therapist to determine the improvement of the special needs individual based on input provided into the special needs module 102. In one embodiment, therapist module 103 is designed to enable the therapist schedule and plan for a day or week on a day-to-day basis. In one embodiment, the therapist module 103 may be configured to assist the therapist to perform the activities of the special needs individual at the school on day-to-day basis. The self-training module 320 may provide an opportunity for the therapist to get abreast with the latest test protocol and methods that were uploaded in the therapy library. The self-training module 320 may allow the therapist to update their accreditation for becoming compliant to use certain tests for special needs individual. Certification of accreditation may be obtained by performing self-evaluation by the therapist using training module 306.

FIG. 4 is an exploded view of the evaluator module 104, according to one embodiment. Particularly, the evaluator module 104 may include a consolidated evaluation module 402, a consolidated observation module 404, an authority regulation module 406, a test implementation module 408, a resource audit module 410, a finance audit module 412, a test standardization module 414, a complaints module 416, a resource allocation module 418, a regulation module 420, a budget module 240, a summary module 228 and assessment module 232, according to one embodiment. The evaluator module 104 may be specifically designed for administering the special needs management system 100. In one embodiment, the evaluator module 104 may also be provided to assist the administrator or the federal authority to manage the special needs management system 100. Also, in one embodiment, the evaluator module 104 may be configured to provide a platform to assist the evaluator to make decisions regarding resources, finance and entity management.

The consolidated evaluation module 402 may be configured to generate consolidated evaluation results. For example, progress of the child William James may be provided as a consolidated report that describes progress of the child William James in the production of the /s/ sound. The consolidated observation module 404 may be configured to generate a report to enables the parent/administrator to understand the observations made by the therapist. The authority regulation module 406 may be configured to check whether the authority providing the therapy is a right authority to provide therapy. The test implementation module 408 may be configured to enable the evaluator to allow approved tests (e.g., approved from the federal authority) to be performed by the therapist.

The resource audit module 410 may be configured to audit the resources with respect to the particular area, for example, a district or a school. The resource audit module 410 may generate a report that specifies the requirements, resources available, and resource required. For example, the resource audit module may generate a report including information of five special need individuals requiring speech therapy, two special need individuals requiring psychotherapy, availability of two speech therapist and non availability of psychologist in a school in a city of Oakland. The administrator based on the report may then take necessary steps to allocate a psychologist for training of the special need individuals requiring psychotherapy. The finance audit module 412 may be configured to account the budget being spent in a district or school. In one embodiment, the finance audit module 412 may generate a report that provides information about money being spent for providing special education during a particular period of time.

The test standardization module 414 may be configured to collect types of tests the therapist are performing and to authorize a standard set of approved tests to be performed by the therapist. The compliant module 416 may be configured to determine whether the tests, therapy, training and the like are in compliance with the federal rules. In a case, if the test, therapy, training and the like is not compliant to the federal rules, then an alert may be generated and communicated to the administrator thereof. The resource allocation module 418 may be configured to support the administrator/evaluator to identify resources and requirements based on the report generated by the resource audit module 410 to allocate the resources based on the requirement. In some embodiments, the resource allocation module 418 may be configured to allocate a resource to the special needs individual for a treatment schedule.

The regulation module 420 may be configured to the tests performed by the therapist. The budget module 240 as described may be configured to enable the administrator to monitor the budget allocated for providing special education in a district or school. The summary module 228, in addition to the function described above, may be configured to generate the summary of the tasks associated with the special education in a district or school.

FIG. 5 is an exploded view of the management module 105, according to one embodiment. Particularly, management module 105 may include the scheduler module 212, the resource allocation module 418, the summary module 228, the treatment audit module 502, the special needs requirement module 504, the budget module 240, the compliance check module 508, the communication module 250, the reporting module 226, the standardized testing module 222, the rating module 510, a new case module 512, the new therapist recruitment module 514, and a compliant module 516, according to one embodiment.

In one embodiment, management module 105 may be configured to manage the special needs management server. The management module 104 as described herein may be configured to manage the activity of the special needs management server 1600 to the therapist, by providing alerts, informing the administrator, informing the parent, generating reports about the progress of a special needs individual. The management module 105 includes several modules to support the functioning of the management module 105. The modules of the management module 105 are described below.

The treatment audit module 502 may be configured to determine the treatments provided by the therapist. The special need requirement module 504 may be configured to determine special need requirement of the individual based on the condition of the individual. For example, based on the tests performed, the therapist determines the requirement such as problem, treatment to be provided, methods to be used, etc. The communication module 250 may be configured to enable communication with parents regarding the training, therapy, progress and the like of the special needs individual. The compliance check module 508 may be configured to check whether all special education schools are conducting therapies, training, test and the like as per the federal regulations. The training compliance module 518 may be configured to check if thethe training being provided is in compliance with the standard training procedures as suggested by the state and national regulatory boards and associations. The rating module 510 may be configured to enable the parents/administrator to rate the performance of the therapist based on the performance of the therapist and the progress of the special need individual. The new case module 512 may be configured to enable the administrator/ therapist to add new special needs individual information into the database. The new therapist recruitment module 514 may be configured to provide information to the administrator to recruit based on the requirement for the special education. The compliant module 516 may be configured to enable the parent to complain about irregularities, improper treatment of the individual, improper training and the like.

The scheduler module 212 of the management module 105 may be configured to schedule routine database maintenance, report generation, schedule alerts and the like. The summary module 228 of the management module 105 may be configured to generate summary of the specific period regarding finance, number of students enrolled, therapist, and the like and to communicate the reports to the appropriate authority through the reporting module 226. The budget module 240 may be configured to allocate and monitor budget being spent in each of school. In some embodiments, the budget module 240 may be configured to generate alerts regarding shortage or requirement of budget and also request for additional budget. The communication module may be configured to enable communication between the management module 105 and other modules. The standardization testing module 222 of the management module 105 may be configured to provide standard instructions to the therapist regarding use of the standardized tests to the special need individual.

FIG. 6 is an exploded view of parent module 106, according to one embodiment. In particular, the parent module 106 may include a homework 602, a feedback module 604, a therapy module 606, a finance module 608, an insurance company module 610, compliance check module 612, an activity module 614, a test module 618, an audit module 620, the communication module 250, the scheduler module 212, the reporting module 226, and the assessment module 232, according to one embodiment.

In one embodiment, the parent module 106 configured to assist parents by providing a platform to communicate with the therapist and to view daily progress of the special need individual. The homework module 602 may be configured to provide the parents with homework assigned by the therapist. In one embodiment, the homework may be practice or exercises provided by the therapist to practice the training provided by the therapist. For example, the therapist may provide a list of words as homework to parents to make their child to practice production of a /s/ sound. The homework may be provided to the parents to see to that continuous effort is made to improve the condition of the individual. Using the example provided above, the words such as “Sat, Sir, Silly, beside, aside, Boston, mass, toes, boss, etc. may be provided with location of the /s/ sound placed at different location and recited with different words. The parent of the individual may be encouraged to make their special needs individuals articulate these words so that the individual slowly picks up the words and overcomes the articulation problem by continuously practicing the exercise even after reaching home. The feedback module 604 may be configured to enable the parents/therapist of the special needs individual to provide feedback based on the progress of the special need individual. In one or more embodiment, the feedback module may provide a template that includes the individual's information and other information and a space for the therapist/parent to input data. The therapy module 606 may enable the parents to view the therapy details that the therapist is administering to the special need individual. The finance module 608 may be configured to track the fees such as special fees, that are payable by the parent. The insurance company module 610 may be configured to communicate with insurance company to provide insurance cover in case of expenses that are required for providing specialized treatment. The compliance check module 612 may enable the parents of the special needs individual to check whether the therapy and methods administered on the individual is complaint to the state and federal rules. The activity module 614 may be configured to assist the parent to input the activities of the special needs individual such as progress, problems caused due to treatment, or any other information pertaining to the special need individual. The test module 618 may enable the parent to administer certain tests that can be performed by the parent to assess the improvement of the special need individual. For example, the parent can request for some tests to test their special needs individual's improvement. Using the example above, the parent can request for additional words to see if the special needs individual actually understands the location of the sound in the word and how to articulate the word with varying location of the sound.

The audit module 620 may be configured to audit the special need individual's progress and developments through the parent account. Any subjective evaluation measure, such as teacher observation, or even promotion from one grade level to the next, are poor measures of special needs individual's progress. Since all of these examples can be influenced based on an individual's thoughts, feelings, or allegiances, they do not accurately measure whether special needs individual has mastered specific skills. Having a teacher's assurance that special needs individual is “really making progress” means nothing if special needs individual's standardized reading comprehension test indicates that special needs individual has still not learned to read. A parent through the audit module 620 may observe these inputs and provide input to the management. The communication module 250 as described above may be configured to enable communication between the therapist and the parent. The scheduler module 212 in addition to assisting the therapist may be configured to enable the parent to schedule a meeting to discuss about their special needs individual. The reporting module 226 as described above may be configured to generate reports based on requests. The assessment module 232 may be configured to assist the parent in determining the improvement of special needs individual based on the data input into the special needs module 102. In one or more embodiments, the function of the assessment module as illustrated in above mentioned figures is to generate a special needs education report for evaluating a milestone development for a special needs individual through calculation based on the input from the therapist, the evaluator, the parent and a manager.

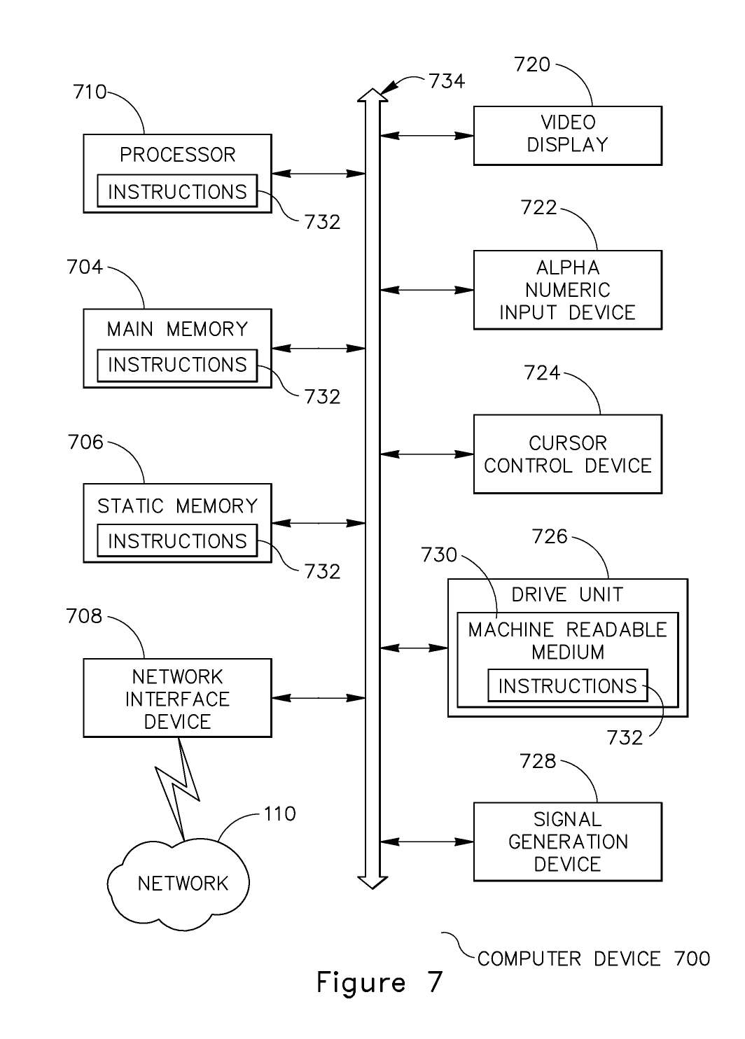

FIG. 7 is a diagrammatic system view 700 of a computer device view in which any of the embodiments disclosed herein may be performed, according to one embodiment. Particularly, the computer system view 700 of FIG. 7 illustrates a processor 702, a main memory 704, a static memory 706, a bus 708, a video display 710, an alpha-numeric input device 712, a cursor control device 714, a drive unit 716, a signal generation device 718, a network interface device 720, a machine readable medium 722, instructions 724, and a network 726, according to one embodiment.

The computer system view 700 may indicate a personal computer and/or a data processing system (e.g., server) in which one or more operations disclosed herein are performed. The processor 702 may be microprocessor, a state machine, an application specific integrated circuit, a field programmable gate array, etc. The main memory 704 may be a dynamic random access memory and/or a primary memory of a computer system. The static memory 706 may be a hard drive, a flash drive, and/or other memory information associated with the computer system. The bus 708 may be an interconnection between various circuits and/or structures of the computer system. The video display 710 may provide graphical representation of information on the data processing system. The alpha-numeric input device 712 may be a keypad, keyboard and/or any other input device of text (e.g., a special device to aid the physically handicapped). The cursor control device 714 may be a pointing device such as a mouse.

The drive unit 716 may be a hard drive, a storage system, and/or other longer term storage subsystem. The signal generation device 718 may be a bios and/or a functional operating system of the data processing system. The network interface device 720 may be a device that may perform interface functions such as code conversion, protocol conversion and/or buffering required for communication to and from a network (e.g., the network 110 of FIG. 1). The machine readable medium 722 may provide instructions on which any of the methods disclosed herein may be performed. The instructions 724 may provide source code and/or data code to the processor 702 to enable any one/or more operations disclosed herein.

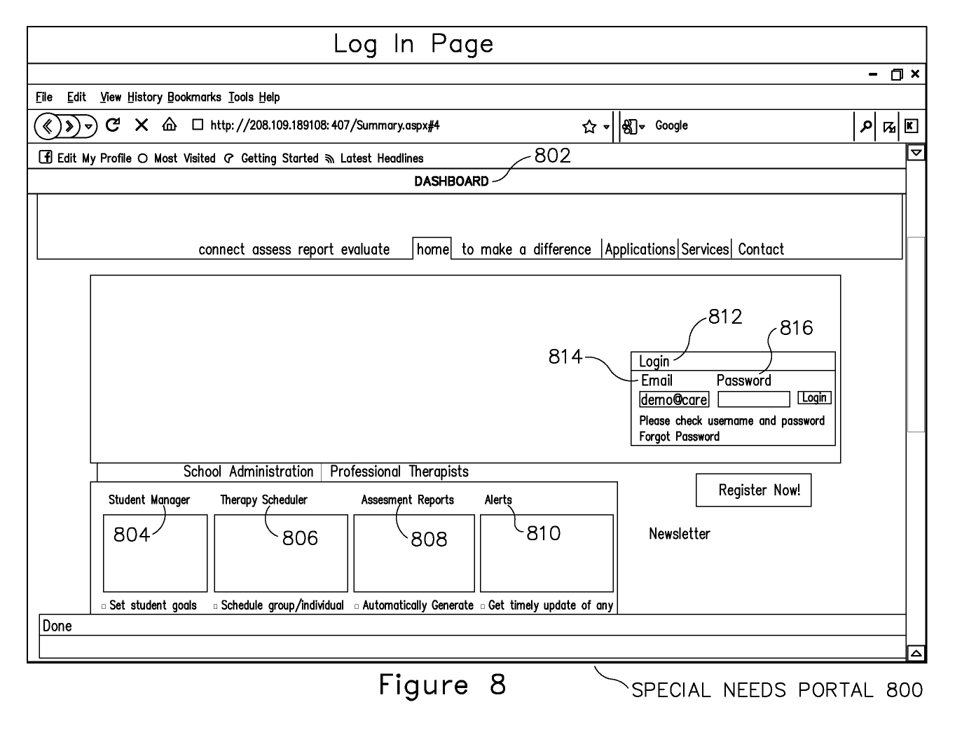

FIG. 8 illustrates a user interface view illustrating a log-in page of special education portal 800, according to one embodiment. In one embodiment, the log-in page may be used by any of the therapist, parents or administrator or users to login to account provided thereof. In one embodiment, the login page may be provided as a user interface in a browser capable of hosting the webpage in the client device which may also be a mobile device.

A dashboard 802 may provide options for the user to input information. A student manager 804 options may be available to the therapist, and the parent to see the activities, history, improvements, stage of treatment, and the like. An assessment report 808 may enable the users to generate an assessment report of therapist, and special needs individual based on privileges provided to the user. For example, the administrator or the evaluator has privilege to generate assessment report of the therapist. An alert 810 may be an option that enables the users to set alerts for future events. For example, the alert 810 option may be helpful to remind the therapist for a treatment schedule for a special need individual. The 812 option illustrates a standard log-in user interface. The log-in 814 and password 816 options are provided to input the log-in information to log-in to their account.

FIG. 9 is a user interface view illustrating a therapist dashboard 900, according to one embodiment. In one embodiment, therapist dashboard 900 may be enabled by the therapist module 103. The therapist dashboard 900 may be configured to enable the therapist to use plan for a day. In the example embodiment, a therapist scheduler 806 may be provided to the therapist to plan for the day. In addition, the user interface view 900 of the example embodiment illustrates additional information such as schools serving, daily logs, and attendance history. Also, in one or more embodiments, the therapist dashboard 900 may provide options for the therapist to change profile information. In one embodiment, the therapist dashboard enabled through the therapist module 103 may also provide alerts and reminders for the therapist about the upcoming schedules through alerts 810 and a workload viewer 904. In one embodiment, with respect to maintaining information about the special need individual, to whom the therapy is to be administered, the therapist dashboard may provide an option to input individual's information including the name/ID or record name or date of birth (DOB) or an unique identification code or number to pull out the individual's information and history from the database 150 through an assessment report generator 908. Further, as per the schedule, the therapy may be provided to the special needs individual and observations, inferences and improvements may be input as the data to the file associated with the individual to the database. The assessment report generator 908 may also enable the therapist to generate assessment report of the special need individual. A student manager 902 option may enable the therapist to access the special need individual's information from the database 150 using individual's name, school,DOB or an unique identification code or number. An additional information may provide recent history and log information associated with recent schedules.

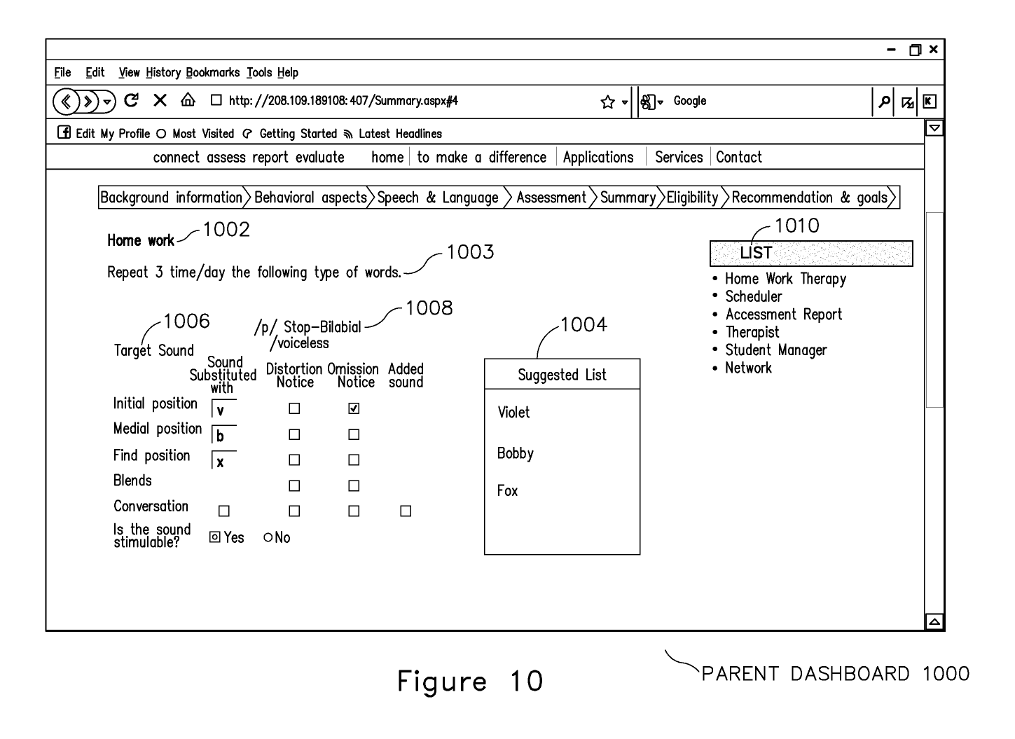

FIG. 10 is a user interface view of a parent dashboard 1000, according to one embodiment. In one embodiment, the parent dashboard 1000 may be configured to enable the parent of the special needs individual to input information about the special needs individual, view the progress of the special needs individual, communicating with the therapist, the users and the like. The parent dashboard 1000 may provide an input form to enable the parent to input the background information of the special needs individual, behavior aspects of the special needs individual, speech and language and inputs on progress of the special needs individual. The assessment section may enable the parent to view the assessment information obtained from the therapist. Also, the parent may also be enabled to provide assessment of the special needs individual through the assessment section. In one embodiment, the homework tab may enable the parent to view the homework 1002 (as illustrated in Figure) assigned by the therapist to the parent. The summary tab may enable the parent to see the treatment summary and other information. The report tab may enable the parent to view the report of the status of their special needs individual and its improvement. In one example embodiment, the report tab may also enable the parent to generate the report of their special needs individual. An eligibility tab allows the parent to submit a request for special needs assistance by filing appropriate forms. Recommendations and goals option may enable the parent to provide recommendations based on parent observations of the therapist.

In one example embodiment, FIG. 10 illustrates a homework 1002 provided by the therapist for the special needs individual to work at home. FIG. 10 suggests parent to make their special needs individual repeat for particular words 1003 at least three times per day. The suggested words by the therapist may be provided in the suggested list 1004. The target sound 1006 may illustrate the position of the letter and stop-bilabial 1008 may be used for observation purposes. The list option 1010 may provide list of options for navigation into various other pages associated with the current working file.

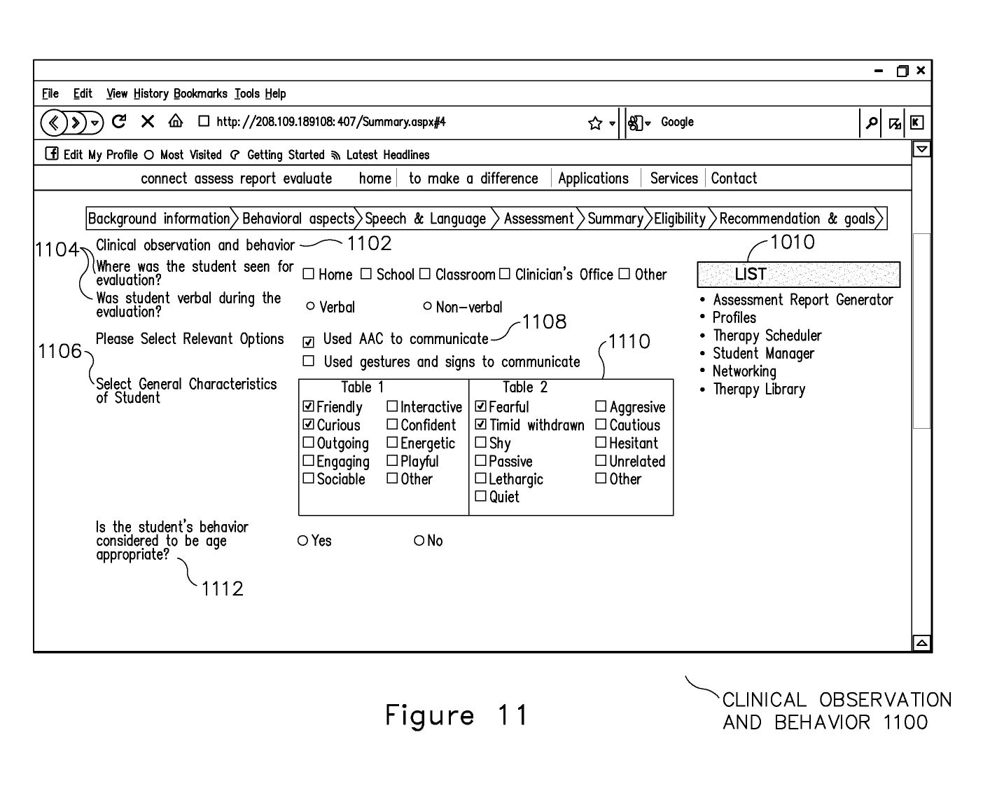

FIG. 11 is a user interface view illustrating a web page for the therapist to input clinical observation and behavior of the special needs individual in a file associated with the individual, according to one example embodiment. In one embodiment, the web page for the GUI would provide a platform for the therapist to input clinical observation and behavior 1102 of the special needs individual with respect to the location, type of evaluation and the like into the webpage provided thereof. In addition, the web page would also enable the therapist to input clinical observations such as general characteristics of special need individual, psychological observation of special needs individual 1102. In one example embodiment, the FIG. 11 illustrates a webpage that provides options for the therapist to input certain general information such as location of evaluation 1104, mode of evaluation 1104, general characteristics of special needs individual 1106 and as to whether the individual's behavior is appropriate to individual's age 1112.

In the example embodiment, location of evaluation 1104 may be a home, school, classroom, clinician's office, or other. The mode of evaluation 1104 may be verbal or non-verbal (observations, reactions to specific contexts). The general characteristics of a student 1106 may provide a table 1110 to enable the therapist easily input the characteristics.

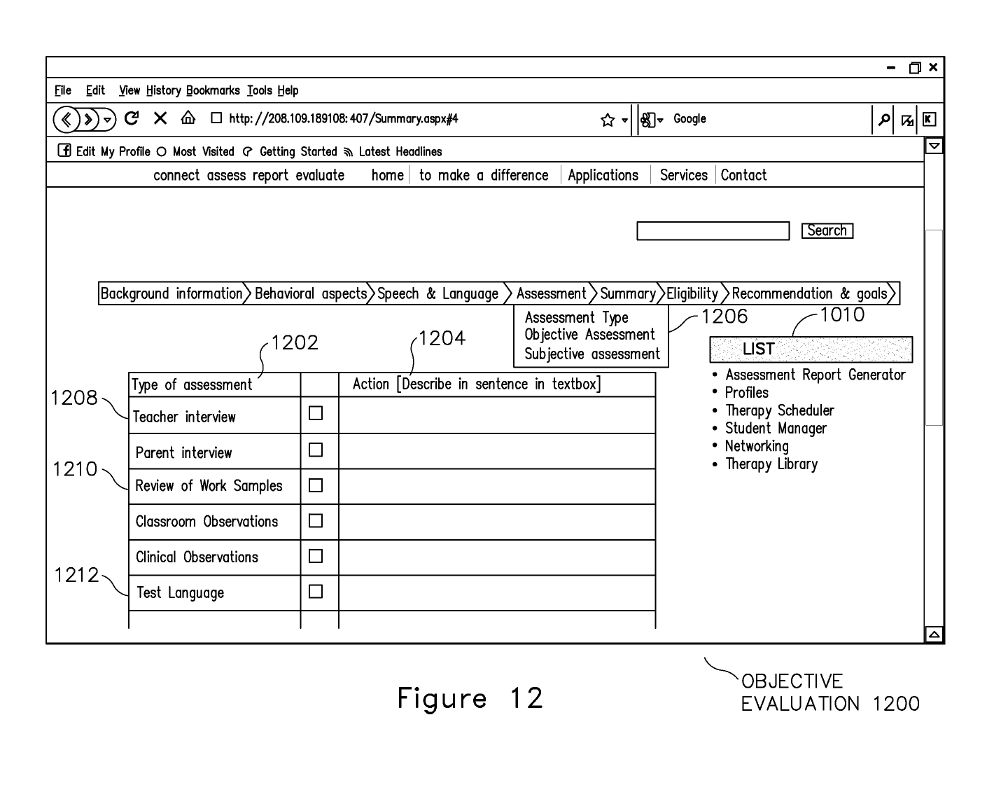

FIG. 12 is a user interface view illustrating a web page for the therapist to input object evaluation 1200 data associated with special need individual, according to one example embodiment. In one embodiment, the special needs management server 150 is also configured to provide a web page to enable the therapist to input objective evaluation data to enable the special needs management server to generate decisions that would aid the therapist to come to certain conclusions associated with the treatment required for special need individual. In one example embodiment, FIG. 12 illustrates an objective evaluation webpage to enable the therapist to input the objective valuation data associated with the special need individual. In example embodiment, FIG. 12 also illustrates type of assessment of the child and the action performed 1202. The type of assessment would include teacher interview, parent interview, review of work samples, classroom observations, clinical observation, and the test language. The action column 1204 provides a text page for the therapist to input objective evaluation data. The assessment type 1206 provides a choice to the therapist to enable the therapist to input objective assessment or subjective assessment. The type of assessment 1202 may include a teacher interview 1208, a parent interview, review of work samples 1210, classroom observations, clinical observations, and the test language 1212.

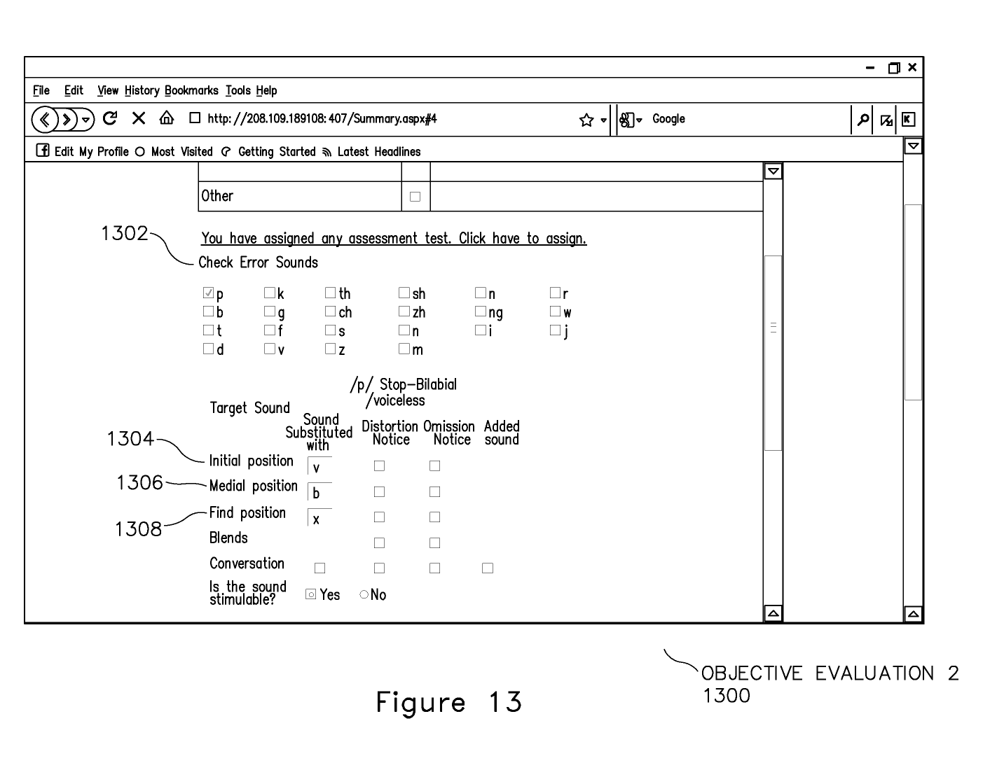

FIG. 13 is a continuation of FIG. 12 illustrating the web page to enable the therapist to input more objective evaluation data, according to one example embodiment. In the example embodiment, FIG. 13 illustrates a web page that enables the therapist to provide an assignment to the parent of the special need individual. In the example embodiment, child may have a speech disorder. The assignment for the parent would include pronunciation exercise for pronouncing a specific letter. In the example embodiment of FIG. 13, the pronunciation exercise would be for the /p/ sound for checking error sounds. Also, in the example embodiment of FIG. 13, target sound table may be provided for the therapist to input the observation of the substitute sound produced by the special needs individual when the therapist provide a specific sound to produce at any of the initial position 1304, a medial position 1306, final position 1308.

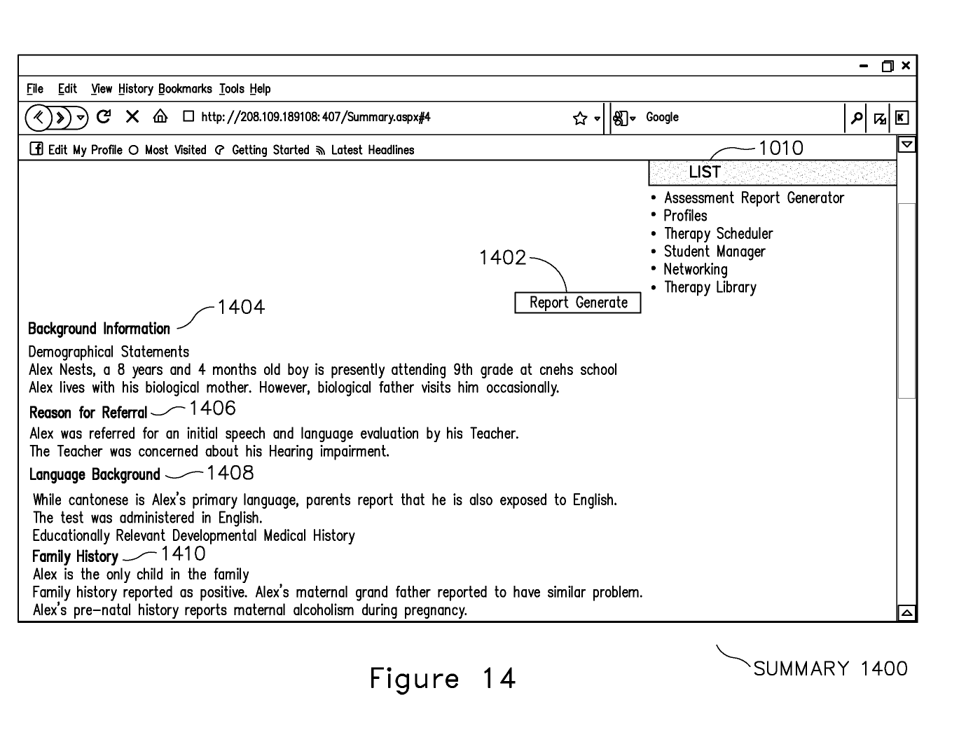

FIG. 14 is a GUI view illustrating the brief summary of special need individual, according to one example embodiment. The brief summary includes background information 1404, a reason for referral 1406, language background 1408, family history 1410, general observations, clinical observations, state of the individual, educationally relevant developmental medical history etc. The summary may provide complete information, complete observation by the therapist, parent and the others. The summary GUI may provide an option to generate a report based on the summary. In one example embodiment, of FIG. 14, the summary GUI illustrates background information 1404 such as age, gender, guardian information and the like. The reason for referral 1406 may provide who and why the individual was referred for therapy. The language background 1408 may provide language that the individual has the ability to understand, and speak. The family history 1410 may provide the family history of the individual.

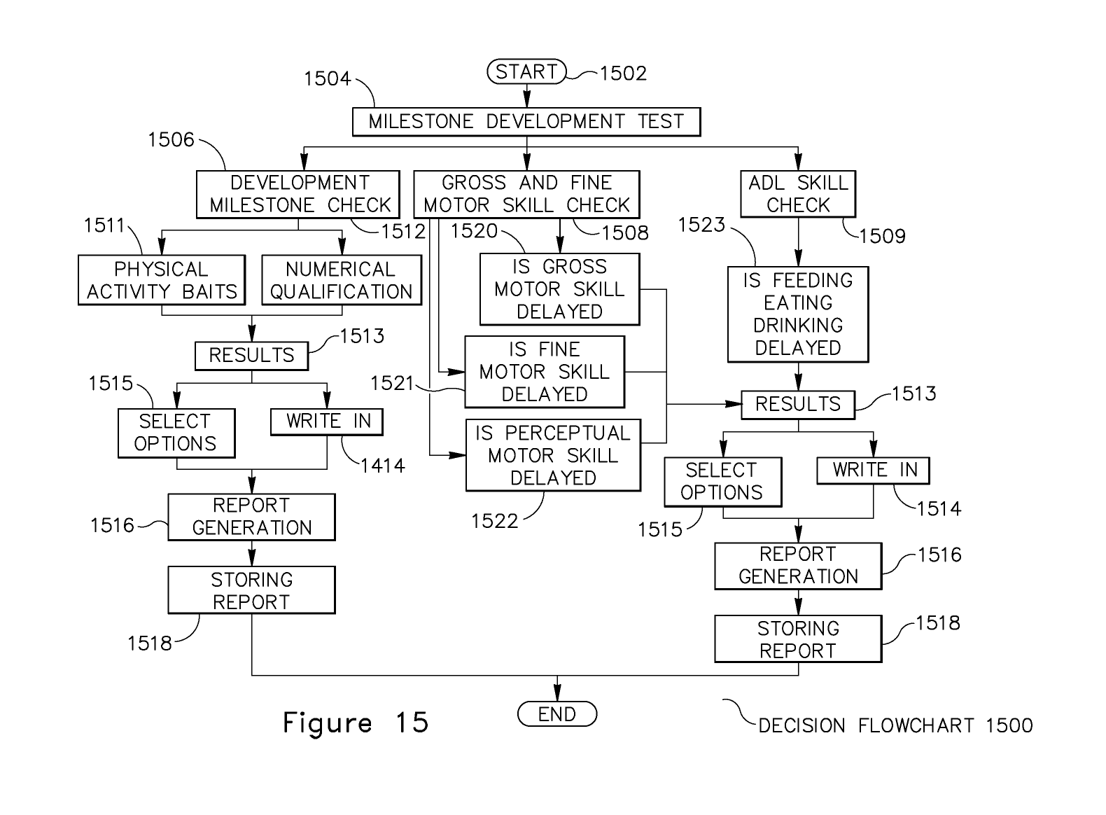

FIG. 15 illustrates a flow chart for providing special education/therapy to a special need individual. In operation 1502, a milestone development test may be performed. In operation 1504, developmental milestone check may be performed. Further, in operation 1506, physical activity traits may be observed and input into the individual's database. In operation 1508, numerical quantification may be performed. In operation 1512, the therapist may be provided with an option to input more observation data and may be provided with an option to write in more therapy information for the progress of the individual. In operation 1516, a report may be generated. In operation 1518, the report may be saved in the individual's file in the database. Similarly, in operation 1522, gross and fine motors skill check of the special needs individual may be performed. In operation 1526, a check may be performed to determine whether there is a delay in fine motors skills of the special need individual. In operation 1530, a check may be performed to determine as to whether there is a delay in perceptual motor skills. Similarly, in operation 1532, Activities of Daily Living (ADL) skill check may be performed. In operation 1534, a check may be performed to determine as to whether there is a delay in feeding, eating, drinking and other basic habits. Further, in operation 1542, a speech milestone check may be performed. In operation 1544, a write in diagram may be generated to determine the therapy to be provided to the special need individual. In operation 1546, based on observations obtained from operations 1522, 1526, 1534, and 1544, a report representing the state of the child may be generated. In operation 1548, the therapist may be provided with an option to input more observation data and to input additional therapy information in operation 1550. In operation 1560, a report may be generated based on operations 1548 and 1550. Further, in operation 1570, the report may be stored in the individual's file in the database.

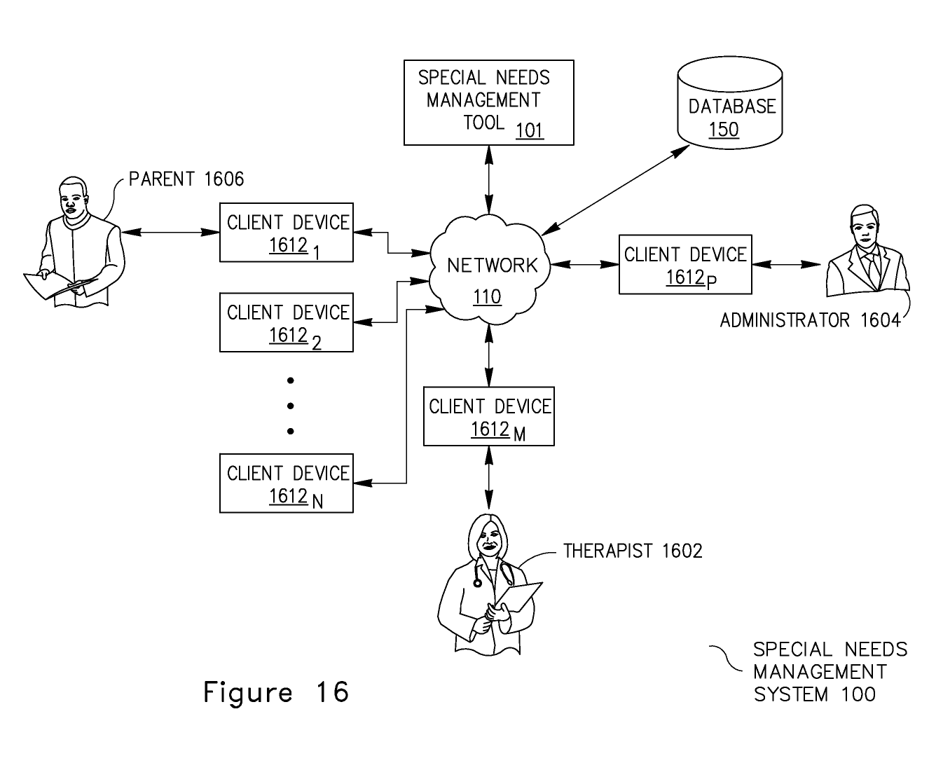

FIG. 16 is a system view illustrating a special educating management system 1650, according to one embodiment. Particularly, FIG. 16 illustrates special needs management server 1600, client device 1612_{1-M} electrically coupled to each other through a network 110, according to one embodiment.

The special needs management system 1650 may be designed to increase productivity of school or clinical staff, efficiency, and accountability. The school or clinical staff as described herein of the school education system 150 may include at least one of therapist and school administrators. The therapist 1614_{1-M} may be teachers who are qualified and trained for training individual (e.g., children) requiring special training The children requiring special training are the ones who may have, but not limited to, one or more problems such as mental retardation, learning disability, speech language impairment, orthopedically impairment, traumatic brain injury, visually impairment, and deafness. The therapist's 1614_{1-M} may be trained personnel trained to train the children with any of aforementioned problems. In one or more embodiments, each of the therapist 1614_{1-M} may be a specialized in providing therapy for one or more problems. The training process may include one or more phases to train the children requiring special needs to enable the children progress from a condition caused due to the problem to a condition where the child can lead a normal social life. The therapist may be any of, but are not limited to physical therapist, occupational therapist, speech language therapist, special teacher, special educator, psychologist, and ABA (Applied Behavior Analysis) therapist.

In one embodiment, the special needs management system 1650 may be designed to reduce work load for the therapist 1614_{1-M}. In one embodiment, the special needs management system 1650 may include the special needs management server 1600 configured to track progress of the special need individual. Also, the special needs management server 1600 may provide a platform for the therapist 1614_{1-M} to input data, individual's condition data, individual's progress data and other formal data. Further, the special needs management server 1600 may enable parents 1608_{1-N} to participate in treatment through providing a platform providing input and communicating with the therapist 1614_{1-M}. Also, the special needs management server 1600 may be configured to provide performance information of the therapist' 1614_{1-M} and progress information of the children who are enrolled for the special education to the administrator 1618 periodically.

The special needs management system 1650 as described herein may be designed to enable communication between the parent's 1608₁₋108_{N}, the therapist 1614_{1-M} and the administrator 1616. In one embodiment, a graphical user interface may be provided for the parent, the therapist and the administrator to input information and to generate reports. For example, an account may be provided to a parent of the special needs individual enrolled for the special education/therapy. The parent may be able to log-in through the account provided thereof, to view schedules for the day, therapy being provided, duration of the therapy, test and progress of the individual. Similarly, each of the therapists 1614₁-1614_{M} may be provided with an account to log-in through the account to create a schedule input a therapy, assessment procedure and inference. Similarly, an administrator (e.g., special director) may also be provided with an account to manage the special needs management system 1650.

The special needs management server 1600 may be any server configured to host the special needs management system 1650. In one embodiment, the special needs management server 1600 may include one or more processors, memory, and database 150 (analogous to database 150 of FIG. 1) associated with the special needs management server 1600 to support the special needs management server 1600. The parents' 1608_{1-N}, the therapist 1614_{1-M} and/or the administrator 1618 may be enabled to input information to the special needs management server 1600 through client device. The client device may be any data processing device such as a computer, a laptop and a mobile device. The communication between each of the client devices and the special needs management server 1600 may be enabled through the network 1606. The special needs management server 1600 may be configured to provide a platform for the special needs management system 1650.

Each special needs individual in public school system may be provided with an individual education plan (IEP) on diagnosing the condition of the individual. The parents are provided with the IEP, the school administrator approves the IEP, and the parents administer the tasks based on IEP. The school needs to comply with the IEP rules. Alerts may be created for the school administrator and the parents periodically. Information of the each of the individual requiring special education may be input into a database 150. In addition, all the progress, therapy information, prescribed therapy lists, history data and the like are stored in the database 150 associated with the special needs management server 1600.

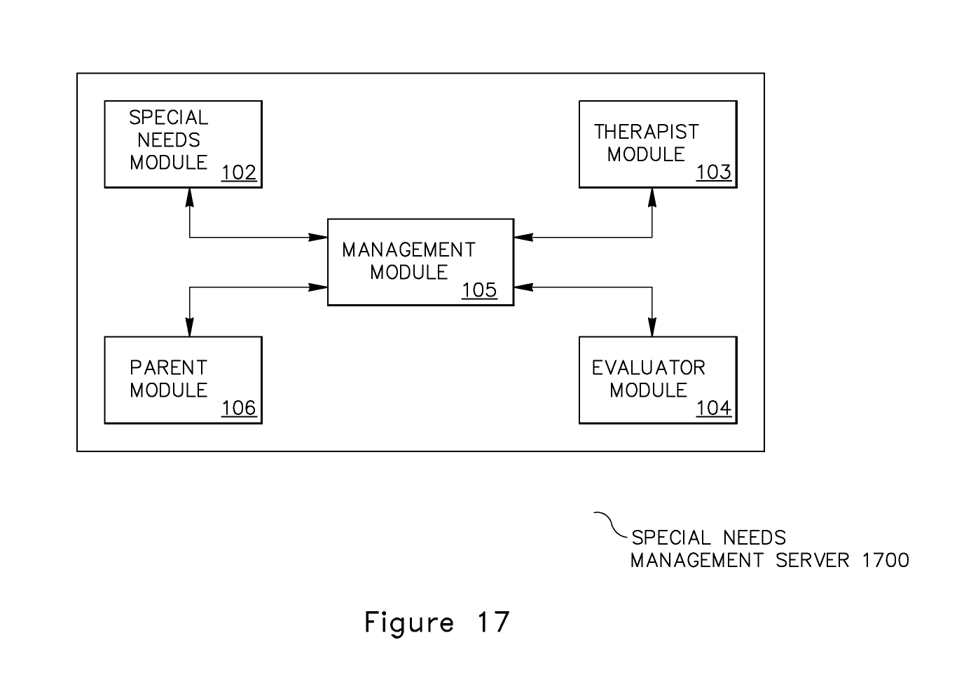

FIG. 17 is an exploded view of the special needs management server 1600 of FIG. 17, according to one or more embodiments. In particular, FIG. 17 includes a special needs module 102, a therapist module 103, an evaluator module 104, management module 105, and a parent module 106, according to one or more embodiments. The special needs module 102 may be configured to implement and manage the special needs management system 1650.

In one embodiment, a special needs individual would visit a hospital, clinic or a school and a physician or the therapist would use the special needs management tool 100 to record the new case. The administrator of the school may allocate and schedule resources using management module 105. The therapist may use the therapist module 103 to provide various services such as generating the special need assessment report, recording the report using the therapist module 103. The evaluator at a higher level of administration can evaluate the services provided to the specials needs individual by using evaluator module 104. The whole system can be made secure by securing the access to comply with regulations.

In addition, it will be appreciated that the various operations, processes, and methods disclosed herein may be embodied in a machine-readable medium and/or a machine accessible medium compatible with a data processing system (e.g., a computer system), and may be performed in any order (e.g., including using means for achieving the various operations). Accordingly, the specification and drawings are to be regarded in an illustrative rather than a restrictive sense.

What is claimed is:

1. A method, comprising:
accessing a special needs management tool using a computer readable medium;
administering a special needs assessment test personalized for testing each of the special needs individual by retrieving the special needs test from a therapy library;
generating a special needs assessment report for a special needs individual using the special needs management tool;
recording the special needs assessment report using the special needs management tool in a real time environment;
storing the special needs assessment report for a review by an authorized personal; and
securing the access to the special needs assessment report based on an access privileged rule.

2. The method of claim 1, wherein the special needs assessment test is generated by an authority and populated to the therapy library, wherein the authorized personal is at least one of a therapist, a physician, a parent, an administrator and a special needs individual.

3. The method of claim 1, wherein the authority is at least one of the physician, the therapist, a government entity, the parent and a managed care personal.

4. The method of claim 1, further comprising:
scheduling a time slot for a care provider to meet the special needs individual on a needs basis; and tracking a milestone development of the special needs individual in the special needs management tool.

5. The method of claim 1, further comprising;
allocating a resource to the special needs individual for a treatment schedule;
selecting a treatment for the special needs individual using the therapy library;
treating the special needs individual using the treatment selected from the therapy library;
evaluating the special needs individual after the treatment; and
recording a treatment result for the special needs individual.

6. The method of claim 5, wherein the therapy library is a compilation of several treatment method and the special needs assessment test suggested by the authority and approved by at least one of a physician, a psychologist, a physiotherapist, an audiologist and a parent.

7. The method of claim 5, further comprising;
updating the therapy library on a periodical basis;
storing a treatment history on a remote location; and
complying with a regulation rule to protect a special needs health record.

8. The method of claim 6, wherein the regulation rule is at least one of medical privacy rules, security privacy rules and special needs individual preference setting rule.

9. The method of claim 1, wherein the special needs management tool is an integrated tool for the therapist, the physician, the management and the parent to view, annotate and store the information for the special needs individual.

10. The method of claim 1, wherein the special needs assessment report is generated by the therapist for the special needs individual.

11. The method of claim 1, wherein the special needs test is at least one for a medical issues, a behavior issue, a developmental issue, a learning disability and a mental health issue.

12. The method of claim 4, wherein the milestone development is a measure of change observed in the special needs individual after therapy has been administered.

13. A system, comprising:
a computer:
a database;
a network; and
a special needs education management tool, wherein the special needs education management tool comprises of a therapist module, an evaluator module, a parent module and a management module.

14. The system of claim 13, further comprising;
an assessment module that calculates the input from the therapist, the evaluator, the parent and a manager to produce a special needs education report for evaluating a milestone development for a special needs individual.

15. The system of claim 13, further comprising:
a memory and at least one processor to execute a set of instructions provided through a special needs module;
the special needs module configured to provide an integrated platform for a therapist, a guardian and an administrator to provide input and to ensure progress of a special needs individual; and
the management module to manage the activity of the special needs management server by at least one of assisting therapist, providing alerts, informing the administrator, informing the parent, generating reports about the progress of a special needs individual.

16. The system of claim 15, wherein the special needs individual is at least one of a hearing impaired, developmentally impaired, visually impaired and speech impaired.

17. A special needs management server, comprising:
a special needs management server configured to implement a special needs management system by providing a platform for a parent, a therapist and an administrator to communicate with each other to improve a condition of a special needs individual; and
at least one client device including a browser to host user interface views provided by the special needs management server.

18. The server of claim 17, further comprising:
a therapist module including a set of modules configured to assist the therapist in at least one of a scheduling, enabling the therapist to input special needs individual information, input clinical and behavioral observation, and to input progress of the special need individual.

19. The server of claim 17, further comprising:
an evaluator module configured to provide a platform to assist the evaluator to make decisions regarding resources, finance and entity management.

20. The server of claim 17, further comprising:
a parent module configured to assist parents by providing a platform to communicate with the therapist and to view daily progress of the individual.
